(19) United States
(12) Patent Application Publication
(10) Pub. No.: US 2025/0160685 A1
(43) Pub. Date: May 22, 2025

(54) PRESSURE BASED PRESENCE MONITORING SYSTEMS AND METHODS OF MONITORING PRESENCE BASED ON PRESSURE

(71) Applicant: Hill-Rom Services, Inc., Batesville, IN (US)

(72) Inventors: Jnanesha Ramegowda, Batesville, IN (US); Aziz Bhai, Batesville, IN (US)

(73) Assignee: Hill-Rom Services, Inc., Batesville, IN (US)

(21) Appl. No.: 18/951,997

(22) Filed: Nov. 19, 2024

Related U.S. Application Data
(60) Provisional application No. 63/600,903, filed on Nov. 20, 2023.

(51) Int. Cl. A61B 5/11; A61B 5/00

(52) U.S. Cl. CPC A61B 5/1115; A61B 5/6892; A61B 5/7282; A61B 5/746; A61B 2562/0247; A61B 2562/046; A61B 2562/168

(57) ABSTRACT

Apparatuses, systems and methods for determining subject positioning are disclosed. An apparatus includes a surface having bladders, pressure sensors fluidly coupled to the bladders, and a controller. The controller is configured to receive an input indicating that a subject is supported on the surface, receive a first signal from each pressure sensor that is indicative of an initial reference pressure of each bladder, receive a subsequent signal from each pressure sensor after a predetermined period of time has elapsed that is indicative of a subsequent pressure of each bladder, determine a rate of change in pressure based on a difference between the first and subsequent signals for each bladder, determine, based on the rate of change for each bladder, that a subject movement has occurred, and determine, based on the subject movement, a positioning of the subject.

### CROSS-REFERENCE TO RELATED APPLICATIONS

This application claims priority to U.S. Provisional Patent Application Ser. No. 63/600,903, filed Nov. 20, 2023, entitled, “PRESSURE BASED PRESENCE MONITORING SYSTEMS AND METHODS OF MONITORING PRESENCE BASED ON PRESSURE,” the entirety of which is incorporated by reference herein.

### TECHNICAL FIELD

The present disclosure relates to monitoring systems and methods and, more particularly, to systems and methods for monitoring subjects on support surfaces.

### BACKGROUND

Certain subject supports, such as hospital beds or the like, have inflatable support surfaces such as mattresses, mattress toppers, etc. on which a person is supported. Such inflatable support surfaces have one or more air bladders that are inflated and deflated to control the pressure in the bladders. The pressure in the bladders correlates to the interface pressure between the skin of the person supported on the surface and the support surface. As a person moves on the bladders, this interface pressure changes, which, in turn, causes the pressure in the bladders to change.

### SUMMARY

In one aspect, a person support apparatus includes a person support surface having a plurality of bladders and a plurality of pressure sensors. Each one of the plurality of pressure sensors is fluidly coupled to a corresponding one of the plurality of bladders. The person support apparatus further includes a controller communicatively coupled to the plurality of pressure sensors. The controller is configured to receive an input corresponding to an indication that a subject is supported on the person support surface and receive a first signal from each one of the plurality of pressure sensors. The first signal is indicative of an initial reference pressure of each of the plurality of bladders. The controller is further configured to receive a subsequent signal from each of the plurality of pressure sensors after a predetermined period of time has elapsed. The subsequent signal is indicative of a subsequent pressure of each of the plurality of bladders. The controller is further configured to determine a rate of change in pressure based on a difference between the first signal and the subsequent signal for each of the plurality of bladders, determine, based on the rate of change for each of the plurality of bladders, that a subject movement has occurred, and determine, based on the subject movement, a positioning of the subject.

In another aspect, a system for determining subject positioning includes a plurality of pressure sensors, each one of the plurality of pressure sensors fluidly coupled to a corresponding one of a plurality of bladders on a person support surface. The system further includes a processor communicatively coupled to the plurality of pressure sensors and a non-transitory storage medium communicatively coupled to the processor. The non-transitory storage medium stores programming instructions thereon that, when executed, cause the processor to receive an input corresponding to an indication that a subject is supported on the person support surface and receive a first signal from each one of the plurality of pressure sensors. The first signal is indicative of an initial reference pressure of each of the plurality of bladders. The programming instructions, when executed, further cause the processor to receive a subsequent signal from each of the plurality of pressure sensors after a predetermined period of time has elapsed. The subsequent signal is indicative of a subsequent pressure of each of the plurality of bladders. The programming instructions, when executed, further cause the processor to determine a rate of change in pressure based on a difference between the first signal and the subsequent signal for each of the plurality of bladders, determine, based on the rate of change for each of the plurality of bladders, that a subject movement has occurred, and determine, based on the subject movement, a positioning of the subject.

In yet another aspect, a method of determining a positioning of a subject includes receiving, by a controller, an input corresponding to an indication that the subject is supported on the person support surface, and receiving, by the controller, a first signal from each of a plurality of pressure sensors communicatively coupled to the controller. The first signal is indicative of an initial reference pressure of each of a plurality of bladders fluidly coupled to a corresponding one of the plurality of pressure sensors. The method further includes receiving, by the controller, a subsequent signal from each of the plurality of pressure sensors after a predetermined period of time has elapsed. The subsequent signal is indicative of a subsequent pressure of each of the plurality of bladders. The method further includes determining, by the controller, a rate of change in pressure based on a difference between the first signal and the subsequent signal for each of the plurality of bladders, determining, based on the rate of change for each of the plurality of bladders, that a subject movement has occurred, and determining, based on the subject movement, the positioning of the subject.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective side view of an illustrative person support system including a person support apparatus according to one or more aspects shown and described herein;

FIG. 2 illustrates a block diagram of illustrative hardware components of a person support system according to one or more aspects shown and described herein;

FIG. 3 schematically illustrates fluid and communication interconnections between various components of a person support system according to one or more aspects shown and described herein.

FIG. 4 illustrates a routine 400 in accordance with one embodiment.

FIG. 5A illustrates an aspect of the subject matter in accordance with one embodiment.

FIG. 5B illustrates an aspect of the subject matter in accordance with one embodiment.

FIG. 5C illustrates an aspect of the subject matter in accordance with one embodiment.

FIG. 5D illustrates an aspect of the subject matter in accordance with one embodiment.

### DETAILED DESCRIPTION

Embodiments described herein are directed to presence sensing systems, person support apparatuses that employ a presence sensing system, and methods of determining presence on a person support apparatus. The presence sensing systems and person support apparatuses described herein generally include a plurality of pressure sensors disposed in or fluidly coupled to associated bladders. The bladders are positioned on or near a support surface of the person support apparatus. The pressure sensors are communicatively coupled to a controller that obtains pressure readings and determines the presence of a subject on the person support apparatus based on a rate of change of pressure within one or more of the bladders. The methods described herein may be embodied as one or more processes completed by a controller to receive pressure data from the pressure sensors, determine a rate of change of the pressure data over a predetermined period of time, and determine whether that rate of change is indicative of movement, positioning, and/or an exit event.

Existing sensing modalities include the use of components such as load cells or other sensors that require specific placement on the person support apparatus, complex installation procedures, complex algorithms in order to accurately obtain and process data from the sensors, and/or the like. As such, it is difficult and costly to implement such modalities. In contrast, the present disclosure, in using air bladders and pressure sensing, can be quickly and easily implemented as either an integrated component or as an add-on component that minimizes complexity and cost associated with implementation. The present disclosure also contemplates utilizing existing components on some person support surfaces (e.g., existing bladders), which further minimizes cost and complexity of implementation. The present disclosure works on the principle of volume gain when a subject weight is removed on an air bladder, which has already been inflated to a predetermined pressure. In addition, the systems described herein can also be adapted to existing microclimate management (MCM) devices (e.g., Hillrom MRS devices offered by Hillrom, Inc. Batesville, IN) since an actual subject weight is not needed for the purposes of determining movement and positioning.

It should be appreciated that, by using a rate of pressure change, the systems and methods described herein can be used despite leakage of pressure from bladders that occur over time. That is, small air leakage from bladders is common, but such pressure decay occurs over a much longer period of time relative to sharp changes in pressure that occur as a result of subject movement. As such, by monitoring not only a pressure change, but a rate of pressure change, the present systems and methods are able to accurately distinguish between pressure changes that occur as a result of leakage and those that occur as a result of subject movement.

Various embodiments of the presence sensing systems, person support apparatuses, and methods for operation of the person support apparatuses are described in more detail herein. Whenever possible, the same reference numerals will be used throughout the drawings to refer to the same or like parts.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

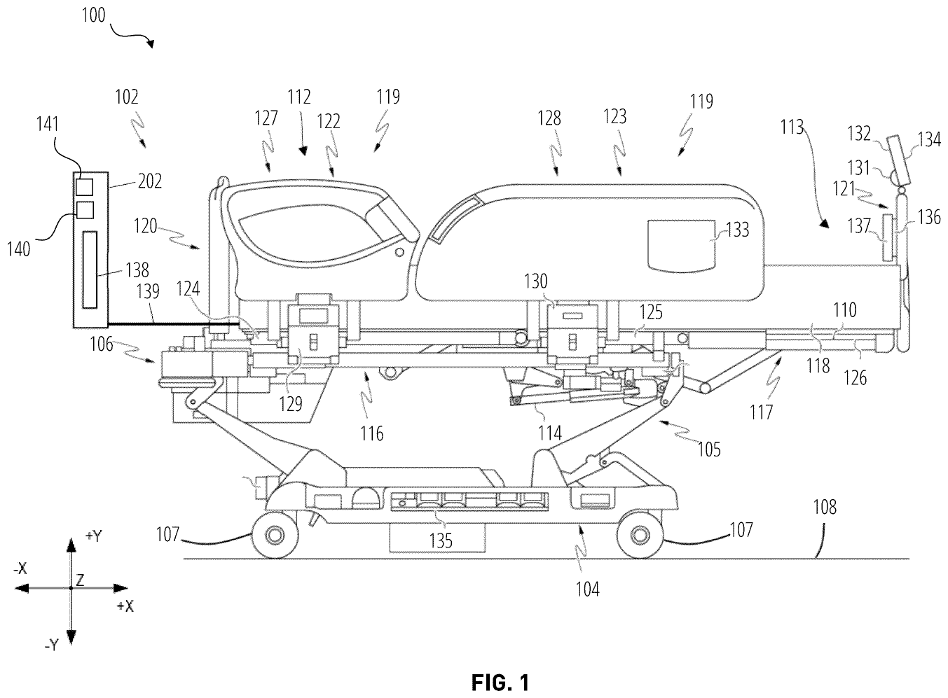

A person support system 100 that includes a person support apparatus 102 according to various embodiments is depicted in FIG. 1. The person support apparatus 102 can be, for example, a hospital bed, a stretcher, a patient lift, a chair, an operating table, or similar support apparatuses commonly found in hospitals, nursing homes, rehabilitation centers or the like. As shown in FIG. 1, the person support apparatus 102 includes a base frame 104, a plurality of lift members 105 coupled to the base frame 104, and an upper frame 106 movably supported by the plurality of lift members 105 above the base frame 104. The base frame 104 includes a plurality of wheels or casters 107 that are movable along a ground surface 108. In various embodiments, the person support apparatus 102 includes a support surface 110 that is supported by the upper frame 106. The support surface 110 has a first section, designated as a head end 112, and a second section, designated as a foot end 113. The support surface 110 may be, for example, a deck 117. The deck 117 includes a head section 124, a seat section 125, and a foot section 126, which engage a bottom surface of a person support surface 118 that is configured to support a subject thereon, as described herein.

The plurality of lift members 105 are coupled to various linear actuators 114 (such as jack motors and the like) and related mechanical and electrical components which facilitate raising, lowering, and tilting of the upper frame 106, and thus the support surface 110, with respect to the base frame 104. Tilting of the support surface 110 relative to the base frame 104 may also be referred to herein as orienting the support surface 110 in a Trendelenburg orientation or a reverse Trendelenburg orientation. In a Trendelenburg orientation, the head end 112 of the support surface 110 is lower than the foot end 113 of the support surface 110 while in a reverse Trendelenburg orientation, the foot end 113 of the support surface 110 is lower that the head end 112 of the support surface 110.

In embodiments, the upper frame 106 can include an upper frame base 116 which supports the deck 117. The person support apparatus 102 may further include side rails 119, a headboard 120, and a footboard 121. The side rails 119, the headboard 120, and the footboard 121 are supported by the upper frame 106, as shown in FIG. 1. The side rails 119, the headboard 120, and the footboard 121 are affixed to the upper frame 106 such that they generally define the perimeter edge of the upper frame 106. In some embodiments, the side rails 119 may include a plurality of sections. For example, in some embodiments, the side rails 119 may each include a head side rail 122, located adjacent to the head end 112 of the support surface 110, and an intermediate side rail 123 positioned between the head side rail 122 and the foot end 113. At least one of the side rails 119 may include a first user interface 133 and a second user interface 134 for control and/or display of the features of the person support apparatus 102. Other user interface components, such as, for example, foot pedal controls 135 may be coupled to the base frame 104 and communicatively coupled to one or more actuators, motors, or the like for raising and lowering a vertical positioning of the upper frame 106 (e.g., movement in the +Y/−Y directions of the coordinate axes depicted in FIG. 1).

As noted above, the upper frame base 116 is coupled to the lift members 105 and supports the support surface 110. In some embodiments, the foot section 126 may be coupled to the upper frame base 116 and moves between an extended position and a retracted position with respect to the upper frame base 116. For example, a linear actuator (not shown) may be coupled to the foot section 126 of the deck 117 in order to enable the length of the person support apparatus 102 to be adjusted. For example, the foot section 126, may be retracted to shorten the length (e.g., along the +X/−X axis of the coordinate axes of FIG. 1) of the person support apparatus 102 or may be extended to lengthen (e.g., along the +X/−X axis of the coordinate axes of FIG. 1) the person support apparatus 102. In embodiments, automatic (e.g., active) extension and retraction of the foot section 126 may be accomplished with the actuator, which may automatically extend and retract the foot section 126 based on signals received from a control unit. In embodiments, the foot section 126 may also be slidable with respect to the upper frame base 116 such that the actuator provides a passive assist in facilitating leg presses. As such, when the subject presses against the foot section 126, the actuator causes extension and sliding of the foot section 126 away from the upper frame base 116.

In some embodiments, the head side rail 122 is coupled to the head section 124 of the deck 117 and the intermediate side rail 123 is coupled to the seat section 125 of the deck 117. The head side rail 122 includes a side rail body 127 and a movement assembly 129. The side rail movement assembly 129 movably couples the side rail body 127 to the upper frame 106 and move the side rail body 127 between a deployed position and a stowed position. When the head side rail 122 is in the deployed position, at least a portion of the head side rail 122 is positioned above the deck 117 and/or the person support surface 118. When the head side rail 122 is in the stowed position, the head side rail 122 is positioned below at least the person support surface 118. In embodiments, the head side rail 122 may also be positioned below the deck 117 when in the stowed position. In some embodiments, the movement assembly 129 includes a locking mechanism (not shown) that maintains the side rail body 127 in the deployed position and/or the stowed position.

The intermediate side rail 123 includes a side rail body 128 and a movement assembly 130. The side rail movement assembly 130 movably couples the side rail body 128 to the upper frame 106 and move the side rail body 128 between a deployed position and a stowed position. When the intermediate side rail 123 is in the deployed position, at least a portion of the intermediate side rail 123 is positioned above the deck 117 and/or the person support surface 118. When the intermediate side rail 123 is in the stowed position, the intermediate side rail 123 is positioned below at least the person support surface 118. In embodiments, the intermediate side rail 123 may also be positioned below the deck 117 when in the stowed position. In some embodiments, the movement assembly 130 includes a locking mechanism (not shown) that maintains the side rail body 128 in the deployed position and/or the stowed position.

The head section 124 and the foot section 126 are each movable relative to the seat section 125. For example, the head section 124 and the foot section 126 pivotally raise and lower relative to the seat section 125. The person support surface 118 is flexible such that it can be articulated along with the support surface 110, as discussed below. The person support apparatus 102 includes one or more motors, actuators, or the like, which, in some embodiments, include linear actuators with electric motors to move the head section 124 and the foot section 126 relative to upper frame 106 and operate the lift members 105 to raise, lower, and/or tilt the upper frame 106 relative to base frame 104. It should be appreciated that these actuators are separate and distinct from the actuators that form the lift members 105 provided between the base frame 104 and the upper frame 106 for moving the upper frame 106 relative to the base frame 104. These actuators are generally understood in the hospital bed art and thus are not illustrated herein. Alternative actuators or motors contemplated by this disclosure include hydraulic cylinders and pneumatic cylinders, for example.

In the illustrative embodiment, the seat section 125 is fixed in position with respect to the upper frame 106 as the deck 117 moves between its various subject supporting positions including a horizontal position, shown in FIG. 1, to support the subject in a supine position, for example, and a chair position, to support the subject in a sitting up position. In embodiments, the seat section 125 moves relative to the base frame 104, such as by pivoting and/or translating the entire deck 117. In embodiments in which seat section 125 pivots and/or translates, the head section 124 and the foot section 126 also move along with seat section 125.

The person support apparatus 102 further includes a plurality of user interfaces. In the embodiment of the person support apparatus 102 shown in FIG. 1, a first user interface 133 is located on an outer surface of one of the intermediate side rails 123. A second user interface 134 is coupled to the footboard 121. However, it should be understood that the user interfaces 133, 134 may be located elsewhere on the person support apparatus 102. In embodiments, the user interface 134 may include a speaker 131 and/or a display 132. Each of the user interfaces 133, 134 includes user interface hardware components such as buttons, joysticks, touchscreens, and other suitable user controls for operating the respective user interface 133, 134. In embodiments, the user interface 134 may be communicatively coupled to a pressure surface 137 which is mounted to the footboard 121 of the person support apparatus. In the embodiment shown in FIG. 1, the footboard 121 is located near the foot end 113 and extends above the support surface 110. The pressure surface 137 may include a sensor 136, such as a flexing force pressure sensor, which measures the amount of force applied to the sensor 136. As noted hereinabove, the second user interface 134 may be in the form of or include a display 132. The display 132 may be a touchscreen, LCD screen, or other suitable display.

In embodiments, the person support apparatus 102 further incudes a fluid supply device 202. The fluid supply device 202 includes a pump 138 for drawing in fluid, for example, air, water, gel, or the like, and a conduit 139 for delivering the fluid from the fluid supply device 202. As such, the conduit 139 interconnects the pump 138 of the fluid supply device 202 to one or more bladders positioned within one or more sections of the deck 117, such as, for example, the head section 124, the seat section 125, and/or the foot section 126, as described in more detail herein. In embodiments, the pump 138 may be a manually operated pump such as, for example, a hand or foot pump. In other embodiments, the pump 138 may be a power operated pump such as, for example, a motorized pump.

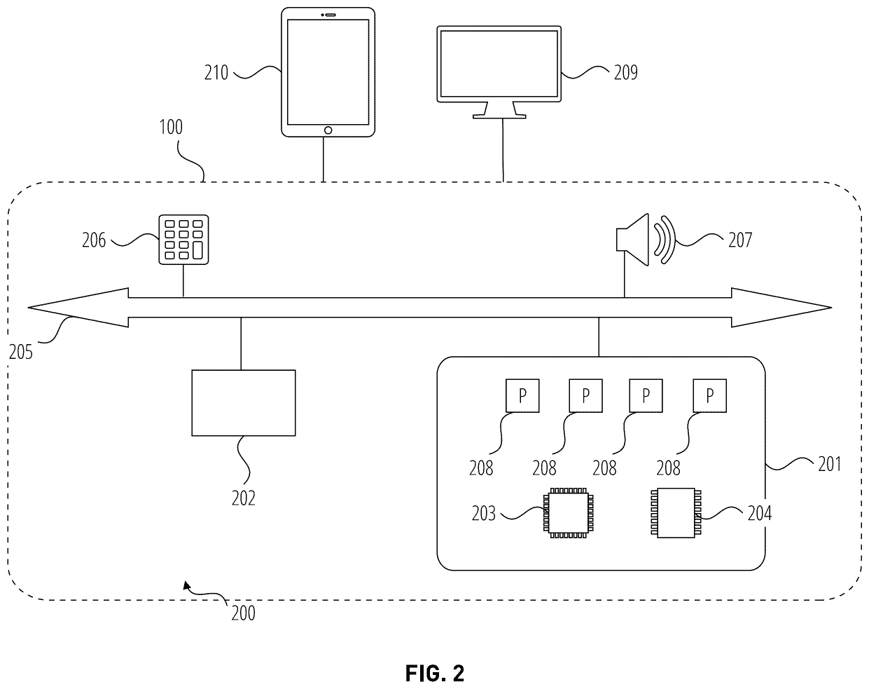

Referring now to FIG. 2, illustrative hardware components 200 of the person support system 100 are illustrated. It should be understood that the person support system 100 may include the person support apparatus 102 (FIG. 1) or may be a standalone system utilized as a retrofit or incorporated onto or into an existing person support assembly.

As shown, in embodiments, the person support system 100 includes the fluid supply device 202 and the controller 201. The controller 201 includes one or more processors 203 and one or more memory modules 204. Each of the one or more processors 203 may be any device capable of executing machine readable and executable instructions. Accordingly, each of the one or more processors 203 may be an integrated circuit, a microchip, a computer, or any other computing device. The one or more processors 203 are coupled to a communication path 205 that provides signal interconnectivity between various modules of the person support system 100. The communication path 205 may communicatively couple any number of processors 203 with one another, and allow the modules coupled to the communication path 205 to operate in a distributed computing environment. Specifically, each of the modules may operate as a node that may send and/or receive data. As used herein, the term “communicatively coupled” means that coupled components are capable of exchanging data signals with one another such as, for example, electrical signals via conductive medium, electromagnetic signals via air, optical signals via optical waveguides, and the like.

The one or more memory modules 204 are non-transitory and may include RAM, ROM, flash memories, hard drives, or any device capable of storing machine readable and executable instructions such that the machine readable and executable instructions can be accessed by the one or more processors 203. The machine readable and executable instructions may include logic or algorithm(s) written in any programming language of any generation (e.108., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 204. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

The communication path 205 may be formed from any medium that is capable of transmitting a signal such as, for example, conductive wires, conductive traces, optical waveguides, or the like. In some embodiments, the communication path 205 may facilitate the transmission of wireless signals, such as WiFi, Bluetooth®, Near Field Communication (NFC) and the like. In addition, the communication path 205 may be formed from a combination of mediums capable of transmitting signals. In one embodiment, the communication path 205 includes a combination of conductive traces, conductive wires, connectors, and buses that cooperate to permit the transmission of electrical data signals to components such as processors, memories, sensors, input devices, output devices, and communication devices. It is noted that the term “signal” means a waveform (e.108., electrical, optical, magnetic, mechanical or electromagnetic), such as DC, AC, sinusoidal-wave, triangular-wave, square-wave, vibration, and the like, capable of traveling through a medium.

As described herein, the controller 201 may be communicatively coupled to the fluid supply device 202. Alternatively, the controller 201 may wirelessly communicate with the fluid supply device 202. Thus, as discussed in more detail herein, the controller 201 may be configured to operate the fluid supply device 202 between an activated state in which the fluid supply device 202 supplies fluid to or draws fluid from the bladders, and a deactivated state in which fluid is not actively supplied to or drawn from the bladders.

In embodiments, the hardware components 200 also includes an input device 206. The input device 206 may be communicatively coupled to the controller 201 and other components of the person support system 100 via the communication path 205. The input device 206 includes one or more controls for operating the hardware components 200 such as, for example, the fluid supply device 202. The one or more controls may be any suitable user operating controls such as, for example, buttons or tactile input on a touchscreen device.

In embodiments, the hardware components 200 also includes an alert component 207 for providing a visual and/or audible output in response to the fluid supply device 202 being operated. The alert component 207 is coupled to the communication path 205 and communicatively coupled to the one or more processors 203. The alert component 207 may include any medium capable of transmitting an optical output such as, for example, a cathode ray tube, light emitting diodes, a liquid crystal display, a plasma display, or the like. In addition, the alert component 207 may be a touchscreen that, in addition to providing optical information, detects the presence and location of a tactile input upon a surface of or adjacent to the display. Accordingly, the alert component 207 may receive mechanical input directly upon the optical output provided by the alert component 207. As such, the input device 206 may be incorporated into the alert component 207. The alert component 207 may also include a speaker for transforming data signals into mechanical vibrations, such as to output audible prompts or audible information.

In addition to the alert component 207 one or more additional external components may also be communicatively coupled to the person support system 100 for the purposes of remote alerting and/or monitoring. For example, FIG. 2 depicts a remote monitoring device 209 and a caretaker device 210 communicatively coupled to the person support system 100. While FIG. 2 depicts these devices as being external and communicatively coupled to the person support system 100, in some embodiments, the remote monitoring device 209 and/or the caretaker device 210 may be components of the person support system 100. The remote monitoring device 209 may be, for example, a computer, display, or the like that can provide information regarding subject positioning, as described herein. For example, the remote monitoring device 209 may be a nurses station device or the like. The caretaker device 210 may be a mobile device or the like that can provide information regarding subject positioning, as described herein. For example, the caretaker device 210 may be a device having a mobile application for caretakers, such as the Voalte Mobile Solution app available from Hillrom (Batesville, IN).

Also depicted in FIG. 2 is a plurality of pressure sensors 208 communicatively coupled to one or more of the other components depicted in FIG. 2 via the communication path 205. The plurality of pressure sensors 208 may each generally be any type of device (e.g., a transducer) that senses a fluid pressure (e.g., a gas pressure, a liquid pressure, etc.) and converts the sensed fluid pressure into an electric signal that is transmitted via the communication path 205 to one or more other devices, such as, for example, the one or more processors 203 of the controller 201. Illustrative examples of pressure sensors 208 that may be utilized include, but are not limited to, a strain gauge, a piezoresistive strain gauge, a capacitive pressure sensor, an electromagnetic pressure sensor, a piezoelectric pressure sensor, an optical pressure sensor, a potentiometric pressure sensor, a force balancing pressure sensor, a resonant pressure sensor, a sensor that incorporates thermal or ionization sensing, a hydrostatic gauge, a piston gauge, a liquid column (manometer), an aneroid gauge, a Bourdon tube, a diaphragm gauge, a magnetic coupling gauge, and/or the like.

Each of the plurality of pressure sensors 208 is associated with at least one bladder (not depicted) for the purposes of determining a pressure of that bladder, as described in greater detail herein. In some embodiments, each of the pressure sensors 208 may be integrated within the bladder associated therewith (e.g., disposed within an interior of the bladder). In other embodiments, each of the pressure sensors 208 may be fluidly coupled to the bladder associated therewith (e.g., coupled in line between the fluid supply device 202 and the bladder). While FIG. 2 depicts four pressure sensors 208, the present disclosure is not limited to such. That is, the hardware components 200 may include fewer than or greater than four pressure sensors 208 without departing from the scope of the present disclosure. Further, each pressure sensor 208 may be associated with one bladder, or may be associated with more than one bladder (e.g., a plurality of bladders may be fluidly coupled to a single pressure sensor 208). Alternatively, a bladder may have more than one pressure sensor 208 associated therewith (e.g., a single bladder may have two pressure sensors 208 associated therewith).

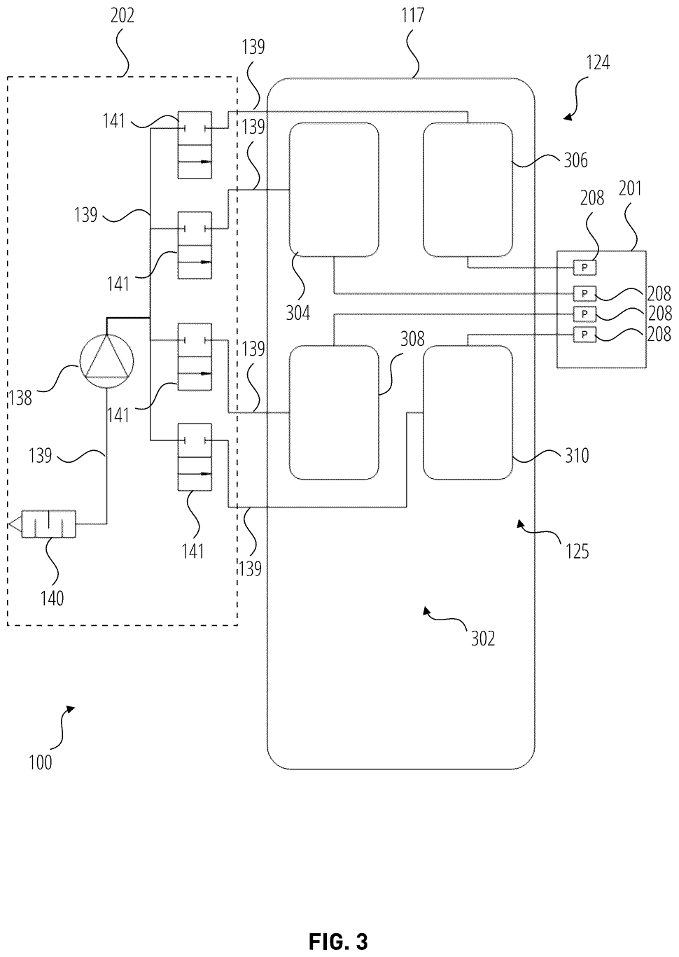

Referring now to FIG. 3, the person support system 100 includes a plurality of bladders 302 positioned above, within, or below the deck 117 of the person support apparatus 102 (FIG. 1), such as, for example, between the person support surface 118 and the deck 117 or on top of the person support surface 118. As described in more detail herein, each of the plurality of bladders 302 may be inflated, such as by the fluid supply device 202, and the pressure of each of the plurality of bladders 302 may be monitored for the purposes of determining a change in pressure over a predetermined period of time so that a subject positioning can be determined therefrom.

As noted above, the plurality of bladders 302 are positioned above, within, or below the deck 117. The plurality of bladders 302 may generally be positioned to correspond to a positioning of a subject supported on the person support surface 118. For example, the head section 124 of the deck 117 may include one or more bladders, such as a first head bladder 304 (e.g., a right head bladder) and/or a second head bladder 306 (e.g., a left head bladder). The first head bladder 304 and the second head bladder 306, in being positioned in the head section 124 of the deck 117, may be such that the head of a subject supported on the person support surface 118 rests on, near, or between the first head bladder 304 and/or the second head bladder 306. In another example, the seat section 125 of the deck 117 may include one or more bladders, such as a first seat bladder 308 (e.g., a right seat bladder) and/or a second seat bladder 310 (e.g., a left seat bladder). The first seat bladder 308 and the second seat bladder 310, in being positioned in the seat section 125 of the deck 117, may be such that the torso, midsection, and/or hips of a subject supported on the person support surface 118 rests on, near, or between the first seat bladder 308 and/or the second seat bladder 310. However, it should be understood that the positioning of the plurality of bladders 302 depicted in FIG. 3 is merely illustrative, and other positions of the plurality of bladders 302 bladders is contemplated and included within the scope of the present disclosure. Further, while FIG. 3 depicts a total of four bladders 302, the present disclosure is not limited to such. That is, the person support system 100 may include fewer than four bladders or greater than four bladders. In some embodiments, the bladders described herein may be bladders that are particularly used for the purposes described herein (e.g., determining subject positioning). In other embodiments, the bladders described herein may be dual purpose bladders that, in addition to the purposes described herein, may further be used for other purposes such as relieving contact pressure between a subject and the person support surface 118, used for positioning or breaking a subject (e.g., rotating a subject, ensuring a subject's spine is particularly positioned), used for therapeutic purposes (e.g., continuous lateral rotation therapy (CLRT) or the like). It should be appreciated that such multi-purpose bladders may be particularly shaped, sized, and/or located for achieving the additional purposes in addition to determining subject positioning as described herein.

As depicted in FIG. 3, each of the plurality of bladders 302 is fluidly coupled to a respective pressure sensor 208. While FIG. 3 depicts the pressure sensors 208 disposed within the controller 201 and coupled via a fluid line (e.g., fluidly coupled) to its corresponding bladder 304-310, the present disclosure is not limited to such. That is, the pressure sensors 208 may be fluidly coupled to their respective bladders 304-310 in other ways. For example, each pressure sensor 208 may be disposed within an interior of a respective bladder 304-310 or fluidly coupled to a supply line that supplies fluid to the corresponding bladder 304-310, as discussed herein. In such embodiments, the pressure sensors 208 are otherwise communicatively coupled to the controller 201 to relay signals corresponding to a sensed pressure to the controller 201 for the purposes of determining a rate of pressure change, as described in greater detail herein.

While FIG. 3 depicts each bladder 304-310 having a respective pressure sensor 208 fluidly coupled thereto, the present disclosure is not limited to such. That is, each bladder 304-310 may be associated with more than one pressure sensor 208 (e.g., a plurality of pressure sensors used to sense pressure in the same bladder) or a single pressure sensor may be fluidly coupled to a plurality of bladders to sense pressure in all bladders fluidly coupled thereto.

Referring to FIGS. 1 and 3, the fluid supply device 202 is generally positioned and configured to supply fluid to each of the plurality of bladders 302. In some embodiments, the fluid supply device 202 may include a fluid source 140 fluidly coupled to each of the plurality of bladders 302 (e.g., via one or more fluid supply lines). While FIG. 3 depicts a single fluid source 140, the present disclosure is not limited to such. That is, the fluid supply device 202 may include more that one fluid source in other embodiments. In some embodiments, the fluid source 140 may be a container housing a fluid to be supplied to the plurality of bladders 302. In some embodiments, the fluid source 140 may be an exterior environment (e.g., not a container).

As described herein, the fluid supply device 202 also includes at least one pump 138 that is fluidly coupled between the fluid source 140 and the plurality of bladders 302. The pump 138 is generally any pump that delivers fluid (e.g., air) to the plurality of bladders 302. Illustrative pumps include, but are not limited to, a centrifugal pump, a diaphragm pump, a rotary vane pump, a gear pump, an axial-flow pump, a piston pump, a positive displacement pump, a peristaltic pump, and/or the like. While FIGS. 1 and 3 depict a single pump 138, the present disclosure is limited to such. That is, in other embodiments, the fluid supply device 202 can include a plurality of pumps 138. For example, each of the plurality of bladders 302 may have a pump 138 associated therewith for pumping fluid to the bladder and/or withdrawing fluid from the bladder. Further, while the pump 138 is depicted as being part of the fluid supply device 202 external to the plurality of bladders 302, the present disclosure is not limited to such. That is, in some embodiments, each of the plurality of bladders 302 may have a pump 138 integrated therewith (e.g., a pump 138 disposed in a membrane that defines an inner cavity of the bladder so as to pump external fluid such as air into the inner cavity).

In embodiments where a single pump 138 is supplying fluid to all of the plurality of bladders 302 (e.g., the embodiment depicted in FIG. 3), the fluid supply device 202 further includes a plurality of valves 141 fluidly positioned between the pump 138 and each of the plurality of bladders 302. That is, the conduits 139 that fluidly couple the pump 138 to the plurality of bladders 302 may incorporate a valve 141 therein or therewith to selectively deliver fluid to an associated one of the plurality of bladders 302. The valves 141 are not limited by this disclosure and may generally be any valve capable of selectively allowing fluid to pass through the conduit 139 to the plurality of bladders 302.

While not specifically depicted in FIG. 3, in some embodiments, the controller 201 may be communicatively coupled to any one of the components of the fluid supply device 202 (e.g., the pump 138, the fluid source 140, the valves 141, etc.) to transmit control signals and/or receive signals therefrom. That is, the controller 201 may control the flow of fluid into or out of the plurality of bladders 302 by transmitting control signals to the various components of the fluid supply device 202. In some embodiments, this control may be due to signals received from the pressure sensors 208 indicative of a pressure in one of the plurality of bladders 302 being too low or too high for a particular purpose. In some embodiments, this control may be for the purposes of moving or rotating the subject, providing therapy (e.g., CLRT or the like), reducing the chances of pressure wounds, and/or the like.

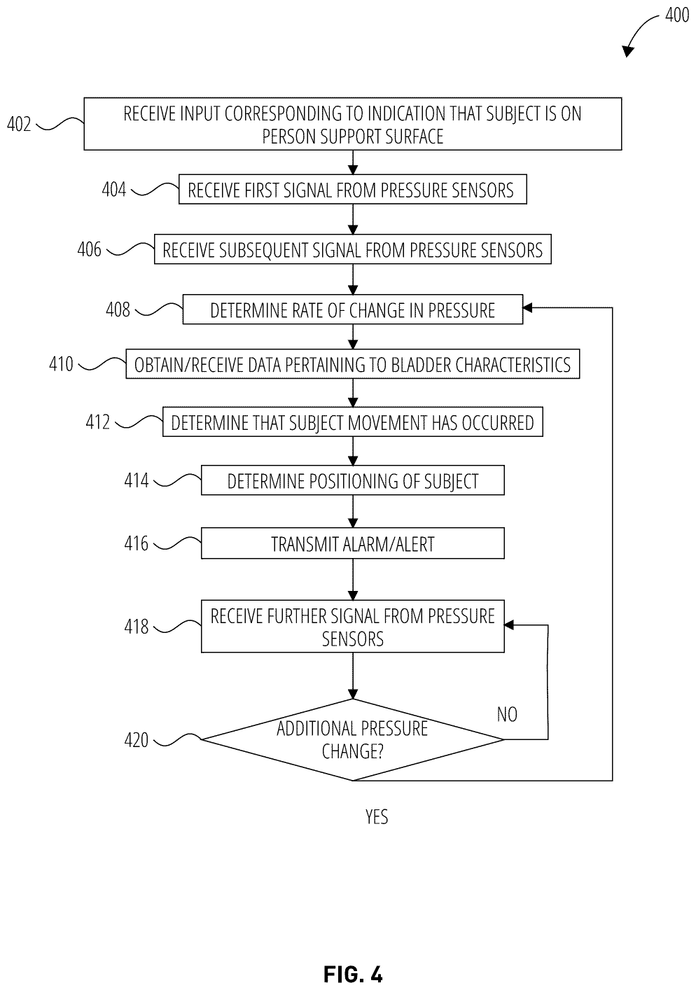

FIG. 4 depicts a flow diagram of an illustrative method of determining a positioning of a subject. The routine 400 depicted in FIG. 4 may generally be completed by the controller 201 (FIG. 2) and/or various components thereof. For example, and with reference to FIG. 2, the routine 400 depicted in FIG. 4 may be embodied as programming instructions within the one or more memory modules 204 and carried out by the one or more processors 203, which may also include the use of various other ones of the hardware components 200 depicted and described herein. While FIG. 4 describes the routine 400 with respect to the controller 201, it should be appreciated that other components may be used and the present disclosure is not limited solely to carrying out the various steps of the routine 400 by the controller 201.

Referring generally to FIGS. 1-4, at block 402, the routine 400 includes receiving, by the controller 201, an input corresponding to an indication that the subject is supported on the person support surface 118. In some embodiments, the input may be automatically generated based on a sensed presence of the subject on the person support surface 118. For example, a sensor such as an image sensor (e.g., a camera, etc.), LIDAR sensor, radar sensor, a force sensor, a load cell, and/or the like may be positioned on or around the person support surface 118 and configured to transmit a signal indicative of a subject supported on the person support surface 118 when sensed. In another example, the pressure sensor 208 may be configured to sense the presence of a subject based on changes in pressure to the plurality of bladders 302 and transmit a signal to the controller 201. That is, the pressure sensor 208, in detecting a change in pressure of at least one of the plurality of bladders 302, provides an indicator of the subject being supported on the person support surface 118. In still another example, the sensor 136, upon receiving an input, may transmit a signal indicative of the presence of a subject on the person support surface 118. In a further example, manipulation of the first user interface 133 and/or the second user interface 134 may be used as the basis for determining the presence of a subject on the person support surface 118, which may result in a signal being sent to the controller 201 at block 402. That is, any interaction with the first user interface 133 and/or the second user interface 134 may result in a signal being sent to the controller 201 at block 402 or alternatively, a user's active actuation of a button or the like in the first user interface 133 and/or the second user interface 134 that is indicative of a presence of the subject (e.g., “subject on bed” button being actuated) may result in a signal being sent to the controller 201 at block 402. In some embodiments, this actuation of a button or the like in the first user interface 133 and/or the second user interface 134 may be referred to as an arming input. In some embodiments, the arming input may be an additional input that is received after receiving the input corresponding to the indication that the subject is supported on the person support surface 118.

At block 404, the routine 400 includes receiving a first signal from each of a plurality of pressure sensors 208. This first signal is generally indicative of an initial reference pressure of each the plurality of bladders 302. That is, in order for a determination of a change in pressure to be determined, the controller 201 uses an initial pressure reading as a baseline. Accordingly, the actions of block 402 and block 404 act to establish a baseline.

As the subject moves on the person support surface 118, the weight of the subject at particular locations causes one or more of the plurality of bladders 302 to compress, which alters the pressure of the fluid contained within that particular bladder. Thus, the person support system 100 described herein can correlate characteristics of the bladder (e.g., shape, size, location, initial pressure, relation to other bladders) and the change in pressure over a particular period of time with particular movements of the subject, and then use those particular movements of the subject to determine subject positioning.

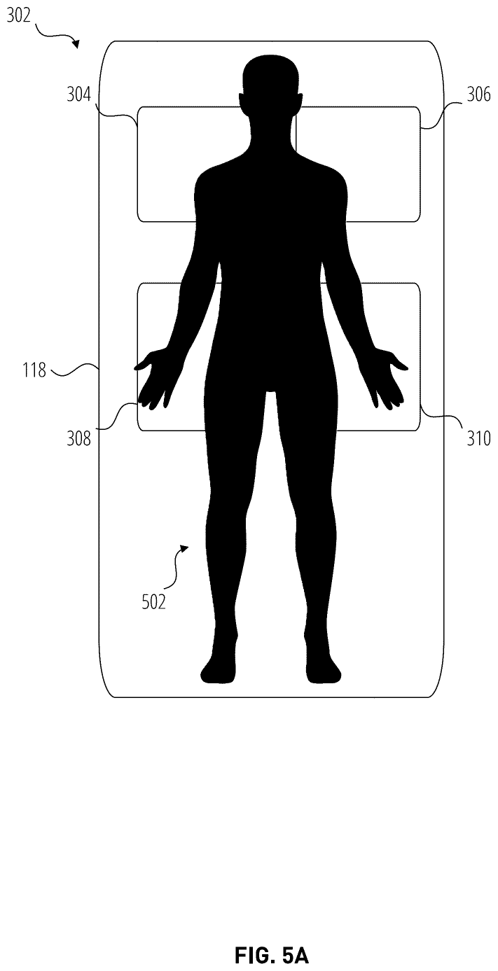

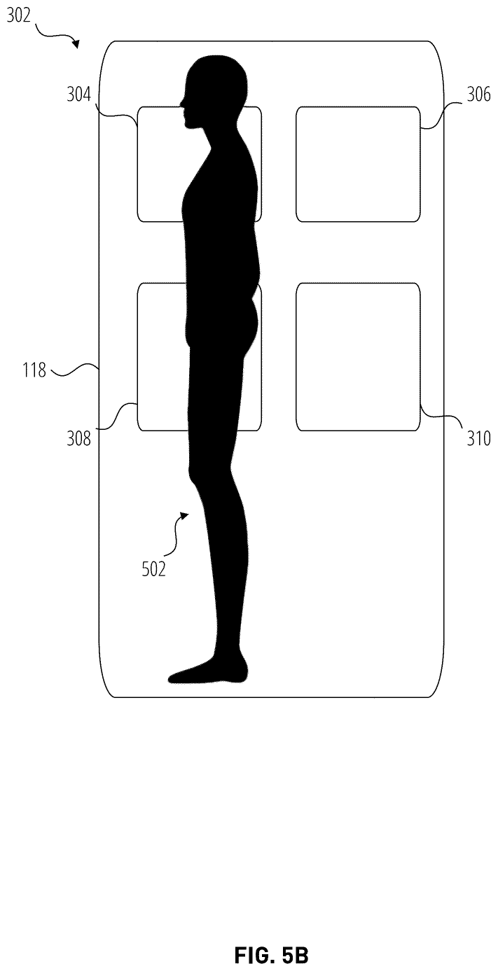

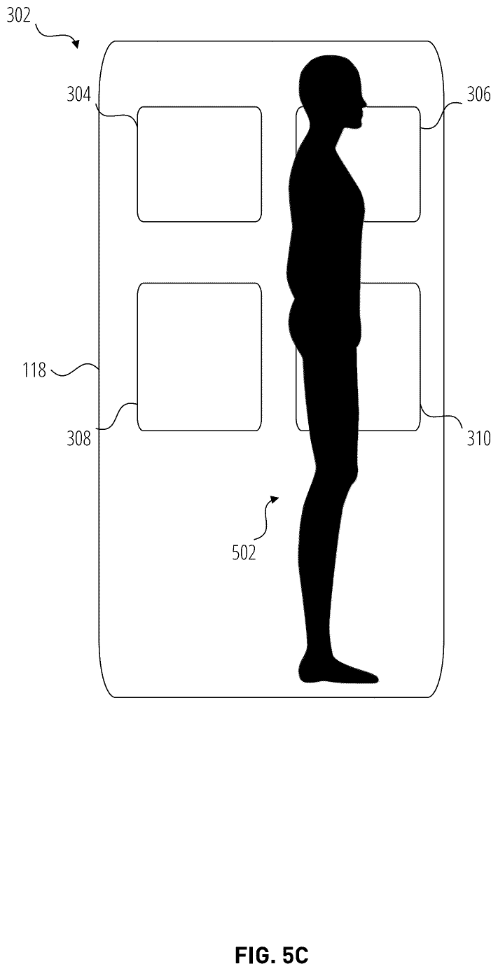

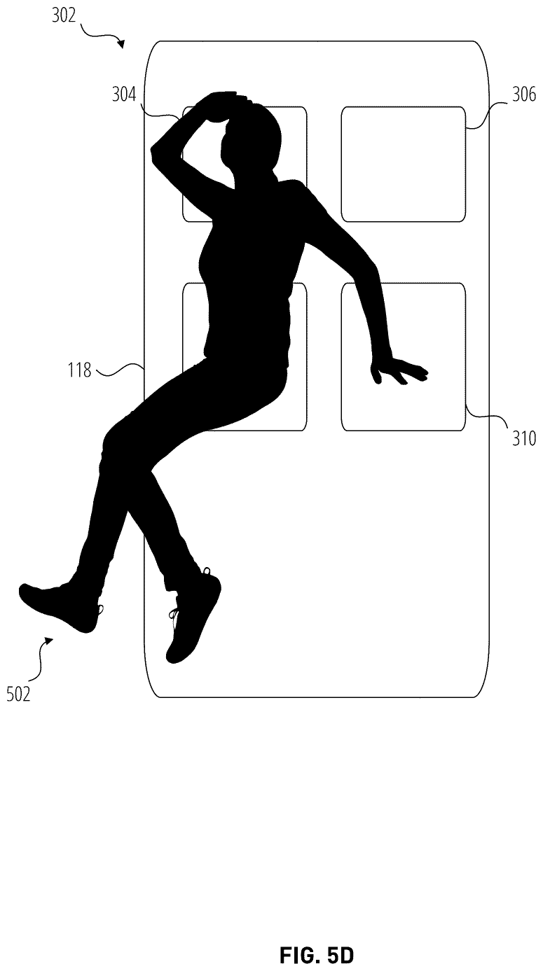

FIGS. 5A-5D depict how illustrative subject positioning causes a change in pressure of each of the plurality of bladders 302. For example, as shown in FIG. 5A, a subject 502 is laying on the person support surface 118 in a typical supine or prone position. In such a positioning, pressure may be generally evenly distributed between the first head bladder 304 and the second head bladder 306 for example, if the subject 502 is laying in a manner such that an equal amount of force is applied to both the first head bladder 304 and the second head bladder 306. Similarly, in such a positioning, pressure may be generally evenly distributed between the first seat bladder 308 and the second seat bladder 310 for example, if the subject is laying in a manner such that an equal amount of force is applied to both the first seat bladder 308 and the second seat bladder 310. However, due to specific positioning of the subject 502 and/or particular anatomical features of the subject 502, an unequal distribution of pressure on the plurality of bladders 302 may be present. However, establishing the initial positioning of the subject and transmitting the first signal from each of the pressure sensor 208 (FIGS. 2-3) associated with each the plurality of bladders 302 in accordance with block 402 and block 404 (FIG. 4) allow for an initial pressure reading to be established for the purposes of determining further movement and positioning of the subject 502, as described herein.

Referring now to FIGS. 5B and 5C, if the subject 502 moves to his/her side (e.g., to the subject's right side as depicted in FIG. 5B or to the subject's left side as depicted in FIG. 5C), the movement causes a change in bladder pressure. For example, as shown in FIG. 5B, movement of the subject 502 to the subject's right side causes a decrease in pressure in the second head bladder 306 and/or the second seat bladder 310, as well as an increase in pressure in the first head bladder 304 and/or the first seat bladder 308. In another example, as shown in FIG. 5C, movement of the subject 502 to the subject's left side causes a decrease in pressure in the first head bladder 304 and/or the first seat bladder 308, as well as an increase in pressure in the second head bladder 306 and/or the second seat bladder 310. These changes in bladder pressure are detected by the pressure sensors 208 (FIGS. 2-3) and are relayed to the controller 201 (FIGS. 2-3) along with the rate of change (e.g., how long it took for pressure to change, how long the pressure change occurred, etc.), which is then correlated by the controller 201 to a particular movement (e.g., by accessing a database of pressure change rates to correlate with the signals received, by utilizing a machine learning model trained to recognize particular pressure change rates as correlating to the particular signals received, and/or the like). Other information, such as the position of the various bladders 302 (including relative positioning), a shape and size of the bladders, or the like may also be utilized to accurately determine subject movement and resultant positioning.

Referring now to FIG. 5D, if the subject 502 attempts to get off the person support surface 118, such movement can cause additional pressure changes in the plurality of bladders 302, which can similarly be utilized for the purposes of determining movement and positioning of the subject 502. For example, the pressure in the second head bladder 306 and the second seat bladder 310 may decrease as the subject 502 rolls off of those bladders, then subsequently a sharp pressure increase may be observed in the first head bladder 304 as the subject 502 puts additional weight on that bladder to lift himself/herself to a sitting position, which is followed by a decrease of pressure in the first head bladder 304 and a corresponding increase of pressure in the first seat bladder 308 as the weight of the subject 502 is shifted to the first seat bladder 308 when the subject 502 sits up. The signals corresponding to these pressure changes, as well as when the changes occur and the rate of change can be transmitted by the pressure sensors 208 to the controller 201, which are then correlated by the controller 201 to a particular movement (e.g., by accessing a database of pressure change rates to correlate with the signals received, by utilizing a machine learning model trained to recognize particular pressure change rates as correlating to the particular signals received, and/or the like). Other information, such as the position of the various bladders 302 (including relative positioning), a shape and size of the bladders, or the like may also be utilized to accurately determine subject movement and resultant positioning. Table 1 below depicts a general correlation of pressures to particular movements as an example:

TABLE 1

| | Pressure | Pressure | Pressure | Pressure | |
|---|---|---|---|---|---|
| | sensor | sensor | sensor | sensor | |
| | (PS) 1 | (PS) | (PS) | (PS) | |
| | (first | 2 (second | 3 | 4 (second | |
| Subject | head | head | (first seat | seat | |
| position | bladder) | bladder) | bladder) | bladder) | Remarks |
| supine/prone | PS1 = | PS2 = | PS3 = | PS4 = | The pressure values |
| position | reference | reference | reference | reference | are substantially |
| | pressure | pressure | pressure | pressure | equal to the |
| | | | | | reference pressure |
| | | | | | (or within an |
| | | | | | acceptable range). |
| | | | | | All bladders |
| | | | | | controlled to same |
| | | | | | reference pressure |
| subject sitting | PS1 < | PS2 < | PS3 > | PS4 > | The change in |
| straight up or | reference | reference | reference | reference | pressure is very |
| HOB up | pressure | pressure | pressure | pressure | rapid and within a |
| | | | | | short period of time |
| Subject | PS1 < | PS2 < | PS3 > | PS4 < | The change in |
| sitting on | reference | reference | reference | reference | pressure is very |
| right side | pressure | pressure | pressure | pressure | rapid and within a |
| | | | | | short period of time |
| Subject | PS1 < | PS2 < | PS3 < | PS4 > | The change in |
| sitting on left | reference | reference | reference | reference | pressure is very |
| side | pressure | pressure | pressure | pressure | rapid and within a |
| | | | | | short period of time |
| Subject out of | PS1 < | PS2 < | PS3 < | PS4 < | The change in |
| the bed | reference | reference | reference | reference | pressure is very |
| | pressure | pressure | pressure | pressure | rapid and within a |
| | | | | | short period of time |

Unlike other systems that also calculate a rate of change in pressure to sense presence of a subject, the present system, in utilizing the plurality of bladders 302 and determining the rate of change of each of the bladders together, the present system can accurately determine a location and a movement of the subject 502 in a way that cannot be accomplished by existing systems. That is, existing systems merely disclose sensing that a change in pressure has occurred, and thus a subject must be located on the bladder (or not on the bladder) in which the change in pressure is sensed. In contrast, by substantially simultaneously obtaining data from a plurality of bladders, together with information about the bladders (e.g., location, relative location, shape, size, initial inflation, etc.), a more accurate representation of how the subject 502 is positioned and moving can be determined, which is not possible from a simple detection of a pressure change in a single bladder or even a simple detection of a pressure change in a plurality of bladders without the additional information described herein.

Referring again to FIGS. 1-4, the routine 400 depicted in FIG. 4 utilizes this rate of change and additional data. More specifically, at block 406, the routine 400 includes receiving a subsequent signal from each of the plurality of pressure sensors 208 after a period of time has elapsed. This subsequent signal is generally indicative of a subsequent pressure of each of the plurality of bladders 302 and is usable as a basis for determining a change in pressure and a rate of change, as described herein. In embodiments, the period of time may be a predetermined period of time. That is, the pressure sensors 208 may be configured to automatically transmit a signal at particular intervals. In such embodiments, the first signal received at block 404 and the subsequent signal received at block 406 may be continuously transmitted at particular intervals so that a change in pressure can be determined together with a rate of change. In other embodiments, the period of time may not be pre-set, but rather the pressure sensors 208 are configured to continuously monitor pressure and transmit a signal whenever a change in pressure has been sensed, along with a signal indicative of the amount of time that elapsed between an initial pressure reading and a subsequent pressure reading of a change in pressure. In either embodiment, the controller 201 receives information pertaining to a change in pressure for each of the plurality of bladders 302 as well as an amount of time in which the pressure change occurred, which can be used to calculate a rate of change and used to determine subject positioning, as described herein.

Accordingly, at block 408, the routine 400 includes determining a rate of change in pressure based on a difference between the first signal and the subsequent signal for each of the plurality of bladders 302. That is, the controller 201 obtains the first signal for each of the plurality of bladders 302, determines the pressure from the first signal for each of the plurality of bladders 302, receives the subsequent signal(s) for each of the plurality of bladders 302, determines the subsequent pressure(s) from the subsequent signal(s), determines the length of time between first and subsequent signals for each of the plurality of bladders 302, and determines the rate of change according to the equation:

$$\frac{dp}{dt}$$

where dp represents the change in pressure and dt represents the change in time.

At block 410, the routine 400 includes obtaining and/or receiving data pertaining to characteristics of the plurality of bladders 302. That is, the controller 201 accesses a database and/or receives inputs pertaining to information that can be used, together with the rates of change of each of the bladders, what movement and positioning of the subject 502 has occurred. Illustrative examples of such data includes, but is not limited to, the location of each of the plurality of bladders 302 on the person support surface 118, the relative location of each of the plurality of bladders 302 with respect to one another, a shape of each of the bladders, a size of each of the bladders, a volume of each of the plurality of bladders 302 (including a max inflate volume), the type of material each of the plurality of bladders 302 is constructed from (e.g., for the purposes of determining elasticity of the material when the bladder is inflated), and/or the like. In some embodiments, block 410 may be optionally included. That is, the routine 400 may also be completed by omitting block 410 and proceeding directly from block 408 to block 412.

At block 412, the routine 400 includes determining that a subject movement has occurred. That is, the controller 201 analyzes the data and determines therefrom that the subject 502 has moved. For example, as described herein, such a determination may be based on a combination of rates of change in pressure of more than one of the plurality of bladders 302, the relative times at which the bladders changed in pressure (e.g., to determine a sequence of bladder pressure change), whether the rate of change indicates an increase in pressure, whether the rate of change indicates a decrease in pressure, and/or the like. Any additional information obtained or received according to block 410 may also be utilized for determining that subject movement has occurred, as described herein. In furtherance of this determination at block 412, the routine 400 continues at block 414 to determine the subject positioning based on the subject movement. That is, referring to FIGS. 5A and 5B for example, if an initial determination that the subject 502 is in a supine positioning in the middle of the person support surface 118 (as indicated by the initial pressure data and depicted in FIG. 5A) and subsequent bladder pressure data indicates a sharp increase in pressure in the first head bladder 304 and the first seat bladder 308, combined with a sharp decrease in pressure in the second head bladder 306 and the second seat bladder 310, then the subject 502 has moved and the positioning correlates to the right side laying position depicted in FIG. 5B.

As the subject 502 positioning and/or movement changes, it may be necessary to transmit one or more alarms and/or alerts in order to notify caretakers, particularly in instances where a subject 502 is not supposed to leave the person support surface 118. As such, at block 416, the routine includes transmitting an alarm and/or an alert. This can be completed in any number or combination of ways, and is not limited by the present disclosure. For example, the controller 201 may signal the alert component 207 to provide an alert, which can then emit a sound, a flashing light, display a video or image, and/or the like. In another example, the controller 201 may transmit an alert to the remote monitoring device 209, such as a device at a nurses station or the like to notify of the movement and/or positioning. In yet another example, the controller 201 may transmit an alert to the caretaker device 210 to notify of the movement and/or positioning. In some embodiments, an alert may only be transmitted if a pre-set alarm setting is activated. That is, if a user actuates an alarm pre-setting on the first user interface 133 or the second user interface 134 (e.g., such that the controller 201 receives an indication of an exit alarm setting), then the controller 201 may determine, based on the determined positioning of the subject 502 on the person support surface 118 as described herein, that a bed exit event is occurring or likely to occur (e.g., that the determined positioning indicates that the subject 502 is sitting up and in a position indicating that a bed exit is about to occur as shown in FIG. 5D for example, or that the determined positioning indicates that the subject 502 is no longer on the person support surface 118). As a result, the controller 201 may transmit an alert or alarm according to block 416 (e.g., transmit a bed exit alarm).

As the routine 400 may continuously monitor for movement and positioning of the subject 502, the routine 400 at block 418 may receive further signals from the pressure sensors 208 as described herein. Those additional signals are used to determine, at decision block 420, whether additional pressure change has occurred. If so, the process returns to block 408 to determine the rate of change in pressure. If not, the process returns to block 418 to continue to monitor for additional signals.

In addition to continuous monitoring, it may occasionally be necessary to re-inflate one or more of the plurality of bladders 302 due to pressure decay that is common in air bladders. As such, in some embodiments, if the pressure of one or more of the plurality of bladders 302 drops below a particular threshold, the controller 201 may transmit a signal to the one or more pumps 138 and/or cause one or more valves 141 to open or close to increase or decrease an internal pressure of one or more of the plurality of bladders 302 as appropriate. This may be accompanied with a process of determining that at least one of the plurality of bladders 302 is overinflated or underinflated, determining an appropriate inflation or deflation command (e.g., determining which pump 138 and/or valve 141 is to be operated, and for how long), and transmit a signal to each pump 138 and/or valve 141 in accordance with the inflation or deflation command.

### Example

Table 2 below depicts an illustrative example of sensed pressures in a plurality of bladders as providing an indication of subject movement. The sensed pressures were obtained from an array of four bladders similar to those depicted in FIGS. 3 and 5A-5D on a Progressa+ bed from Hillrom (Batesville, IN). A simulated subject weighing about 210 pounds was used.

TABLE 2

| | Right head | Left head | Right seat | Left seat | |
|---|---|---|---|---|---|
| | bladder | bladder | bladder | bladder | |
| Subject | (inches | (inches | (inches | (inches | |
| position | H₂O) | H₂O) | H₂O) | H₂O) | Comments |
| Pressure at | 3.9 | 3.7 | 3.9 | 3.8 | Initial pressure |
| start up | | | | | before subject |
| | | | | | placement |
| Pressure | 9.2 | 9.3 | 9.6 | 9.4 | Reference pressure |
| after subject | | | | | |
| ingress | | | | | |
| Subject | 0.5 | 0.2 | 17.8 | 17.8 | Pressure on head |
| sitting | | | | | bladders sharply |
| straight up | | | | | decreases and seat |
| or HOB up | | | | | bladders sharply |
| | | | | | increases |
| Subject | 0.39 | 0.9 | 14.53 | 5.2 | |
| sitting on | | | | | |
| right side | | | | | |
| Subject out | 0.37 | 1.01 | 0.06 | 2.6 | |
| of bed | | | | | |

It should now be understood that the apparatuses, systems, and methods described herein include a plurality of bladders disposed on, in, or under a person support surface are monitored for pressure, and a combination of the rate of change in pressure together with various characteristics of the bladders can be used to determine a positioning and a movement of a subject supported on the person support surface. The apparatuses, systems, and methods described herein can be simple and cost effective to implement, and allow for subject monitoring to avoid issues that may arise with certain subjects that attempt to move to a position that is contraindicated or attempt to leave the person support surface.

To the extent not already described, the different features and structures of the various aspects can be used in combination, or in substitution with each other as desired. That one feature is not illustrated in all of the examples is not meant to be construed that it cannot be so illustrated, but is done for brevity of description. Thus, the various features of the different aspects can be mixed and matched as desired to form new aspects, whether or not the new aspects are expressly described. All combinations or permutations of features described herein are covered by this disclosure.

This written description uses examples to describe aspects of the disclosure described herein, including the best mode, and also to enable any person skilled in the art to practice aspects of the disclosure, including making and using any devices or systems and performing any incorporated methods. The patentable scope of aspects of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Further aspects are provided by the subject matter of the following clauses:

In one aspect, a person support apparatus, includes a person support surface includes a plurality of bladders. The person support apparatus also includes a plurality of pressure sensors, each one of the plurality of pressure sensors fluidly coupled to a corresponding one of the plurality of bladders. The person support apparatus also includes a controller communicatively coupled to the plurality of pressure sensors, the controller configured to receive an input corresponding to an indication that a subject is supported on the person support surface, receive a first signal from each one of the plurality of pressure sensors, the first signal indicative of an initial reference pressure of each of the plurality of bladders, receive a subsequent signal from each of the plurality of pressure sensors after a predetermined period of time has elapsed, the subsequent signal indicative of a subsequent pressure of each of the plurality of bladders, determine a rate of change in pressure based on a difference between the first signal and the subsequent signal for each of the plurality of bladders, determine, based on the rate of change for each of the plurality of bladders, that a subject movement has occurred, and determine, based on the subject movement, a positioning of the subject.

The person support apparatus according to any preceding clause, where the plurality of bladders includes a first head bladder, a second head bladder, a first seat bladder, and a second seat bladder.

The person support apparatus according to any preceding clause, where the controller is further configured to receive data corresponding to a shape and a size of each of the plurality of bladders, and determining that the subject movement has occurred is further based on the shape and the size of each of the plurality of bladders.

The person support apparatus according to any preceding clause, where the controller is further configured to receive an arming input after receiving the input corresponding to the indication that the subject is supported on the person support surface and prior to receiving the first signal.

The person support apparatus according to any preceding clause, where receiving the input corresponding to the indication that the subject is supported on the person support surface includes receiving an arming input.

The person support apparatus according to any preceding clause, where receiving the input corresponding to the indication that the subject is supported on the person support surface includes detecting a change in pressure of at least one of the plurality of bladders that is indicative of the subject being supported on the person support surface.

The person support apparatus according to any preceding clause, further includes an alert component.

The person support apparatus according to any preceding clause, where the controller is further configured to signal the alert component to provide an alert.

The person support apparatus according to any preceding clause, where the controller is further configured to transmit an alert to a remote monitoring device.

The person support apparatus according to any preceding clause, where the controller is further configured to transmit an alert to a caretaker device.

The person support apparatus according to any preceding clause, where the controller is further configured to receive an indication of an exit alarm setting, determine, based on the positioning of the subject, that a bed exit event is occurring or likely to occur, and transmit a bed exit alarm.

The person support apparatus according to any preceding clause, further comprising: a fluid source; and one or more pumps fluidly coupling the fluid source to the plurality of bladders, the one or more pumps communicatively coupled to the controller, wherein the controller is further configured to transmit a signal to the one or more pumps to increase or decrease an internal pressure of each of the plurality of bladders.

The person support apparatus according to any preceding clause, wherein the controller is further configured to determine that at least one of the plurality of bladders is overinflated or underinflated, determine an inflation or deflation command, and transmit the signal to the one or more pumps in accordance with the inflation or deflation command.

The person support apparatus according to any preceding clause, wherein the positioning of the subject is selected from a laying position, an upright sitting position, a right side sitting position, and a left side sitting position.

The person support apparatus according to any preceding clause, wherein the person support surface is a mattress topper positioned over one or more bed components.

The person support apparatus according to any preceding clause, wherein the person support surface is an integrated support surface.

A system for determining subject positioning, the system comprising: a plurality of pressure sensors, each one of the plurality of pressure sensors fluidly coupled to a corresponding one of a plurality of bladders on a person support surface; a processor communicatively coupled to the plurality of pressure sensors; and a non-transitory storage medium communicatively coupled to the processor, the non-transitory storage medium storing programming instructions thereon that, when executed, cause the processor to: receive an input corresponding to an indication that a subject is supported on the person support surface, receive a first signal from each one of the plurality of pressure sensors, the first signal indicative of an initial reference pressure of each of the plurality of bladders, receive a subsequent signal from each of the plurality of pressure sensors after a predetermined period of time has elapsed, the subsequent signal indicative of a subsequent pressure of each of the plurality of bladders, determine a rate of change in pressure based on a difference between the first signal and the subsequent signal for each of the plurality of bladders, determine, based on the rate of change for each of the plurality of bladders, that a subject movement has occurred, and determine, based on the subject movement, a positioning of the subject.

The system according to any preceding clause, wherein the plurality of bladders comprises a first head bladder, a second head bladder, a first seat bladder, and a second seat bladder.

The system according to any preceding clause, wherein the programming instructions, when executed, further cause the processor to: receive data corresponding to a shape and a size of each of the plurality of bladders.

The system according to any preceding clause, wherein the programming instructions that, when executed, cause the processor to determine that the subject movement has occurred is further based on the shape and the size of each of the plurality of bladders.

The system according to any preceding clause, wherein the programming instructions, when executed, further cause the processor to: receive an arming input after receiving the input corresponding to the indication that the subject is supported on the person support surface and prior to receiving the first signal.

The system according to any preceding clause, wherein the programming instructions that, when executed, cause the processor to receive the input corresponding to the indication that the subject is supported on the person support surface comprise receiving an arming input.

The system according to any preceding clause, wherein the programming instructions that, when executed, cause the processor to receive the input corresponding to the indication that the subject is supported on the person support surface comprise detecting a change in pressure of at least one of the plurality of bladders that is indicative of the subject being supported on the person support surface.

The system according to any preceding clause, further comprising an alert component.

The system according to any preceding clause, wherein the programming instructions further cause the processor to signal the alert component to provide an alert.

The system according to any preceding clause, wherein the programming instructions that further cause the processor to transmit an alert to a remote monitoring device.

The system according to any preceding clause, wherein the programming instructions further cause the processor to transmit an alert to a caretaker device.

The system according to any preceding clause, wherein the programming instructions, when executed, further cause the processor to: receive an indication of an exit alarm setting; determine, based on the positioning of the subject, that a bed exit event is occurring or likely to occur; and transmit a bed exit alarm.

The system according to any preceding clause, further comprising: a fluid source; and one or more pumps fluidly coupling the fluid source to the plurality of bladders, the one or more pumps communicatively coupled to the controller, wherein the programming instructions, when executed, further cause the processor to transmit a signal to the one or more pumps to increase or decrease an internal pressure of each of the plurality of bladders.

The system according to any preceding clause, wherein the programming instructions, when executed, further cause the processor to determine that at least one of the plurality of bladders is overinflated or underinflated, determine an inflation or deflation command, and transmit the signal to the one or more pumps in accordance with the inflation or deflation command.

The system according to any preceding clause, wherein the positioning of the subject is selected from a laying position, an upright sitting position, a right side sitting position, and a left side sitting position.

A method of determining a positioning of a subject, the method comprising: receiving, by a controller, an input corresponding to an indication that the subject is supported on the person support surface; receiving, by the controller, a first signal from each of a plurality of pressure sensors communicatively coupled to the controller, the first signal indicative of an initial reference pressure of each of a plurality of bladders fluidly coupled to a corresponding one of the plurality of pressure sensors; receiving, by the controller, a subsequent signal from each of the plurality of pressure sensors after a predetermined period of time has elapsed, the subsequent signal indicative of a subsequent pressure of each of the plurality of bladders; determining, by the controller, a rate of change in pressure based on a difference between the first signal and the subsequent signal for each of the plurality of bladders; determining, based on the rate of change for each of the plurality of bladders, that a subject movement has occurred; and determining, based on the subject movement, the positioning of the subject.

The method according to any preceding clause, further comprising receiving data corresponding to a shape and a size of each of the plurality of bladders, wherein determining that the subject movement has occurred is further based on the shape and the size of each of the plurality of bladders.

The method according to any preceding clause, further comprising receiving an arming input after receiving the input corresponding to the indication that the subject is supported on the person support surface and prior to receiving the first signal.

The method according to any preceding clause, wherein receiving the input corresponding to the indication that the subject is supported on the person support surface comprises receiving an arming input.

The method according to any preceding clause, wherein receiving the input corresponding to the indication that the subject is supported on the person support surface comprises detecting a change in pressure of at least one of the plurality of bladders that is indicative of the subject being supported on the person support surface.

The method according to any preceding clause, further comprising signaling an alert component to provide an alert.

The method according to any preceding clause, further comprising transmitting an alert to a remote monitoring device.

The method according to any preceding clause, further comprising transmitting an alert to a caretaker device.

The method according to any preceding clause, further comprising: receiving an indication of an exit alarm setting; determining, based on the positioning of the subject, that a bed exit event is occurring or likely to occur; and transmitting a bed exit alarm.

The method according to any preceding clause, further comprising: transmitting a signal to the one or more pumps to increase or decrease an internal pressure of each of the plurality of bladders.

The method according to any preceding clause, further comprising: determining that at least one of the plurality of bladders is overinflated or underinflated; determining an inflation or deflation command; and transmitting a signal to one or more pumps in accordance with the inflation or deflation command.

What is claimed is:

1. A person support apparatus, comprising:
a person support surface comprising a plurality of bladders;
a plurality of pressure sensors, each one of the plurality of pressure sensors fluidly coupled to a corresponding one of the plurality of bladders; and
a controller communicatively coupled to the plurality of pressure sensors, the controller configured to:
receive an input corresponding to an indication that a subject is supported on the person support surface,
receive a first signal from each one of the plurality of pressure sensors, the first signal indicative of an initial reference pressure of each of the plurality of bladders,
receive a subsequent signal from each of the plurality of pressure sensors after a predetermined period of time has elapsed, the subsequent signal indicative of a subsequent pressure of each of the plurality of bladders,
determine a rate of change in pressure based on a difference between the first signal and the subsequent signal for each of the plurality of bladders,
determine, based on the rate of change for each of the plurality of bladders, that a subject movement has occurred, and
determine, based on the subject movement, a positioning of the subject.

2. The person support apparatus according to claim 1, wherein the plurality of bladders comprises a first head bladder, a second head bladder, a first seat bladder, and a second seat bladder.

3. The person support apparatus according to claim 1, wherein:
the controller is further configured to receive data corresponding to a shape and a size of each of the plurality of bladders, and
determining that the subject movement has occurred is further based on the shape and the size of each of the plurality of bladders.

4. The person support apparatus according to claim 1, wherein the controller is further configured to receive an arming input after receiving the input corresponding to the indication that the subject is supported on the person support surface and prior to receiving the first signal.

5. The person support apparatus according to claim 1, wherein receiving the input corresponding to the indication that the subject is supported on the person support surface comprises receiving an arming input.

6. The person support apparatus according to claim 1, wherein receiving the input corresponding to the indication that the subject is supported on the person support surface comprises detecting a change in pressure of at least one of the plurality of bladders that is indicative of the subject being supported on the person support surface.

7. The person support apparatus according to claim 1, further comprising an alert component.

8. The person support apparatus according to claim 7, wherein the controller is further configured to signal the alert component to provide an alert.

9. The person support apparatus according to claim 1, wherein the controller is further configured to transmit an alert to a remote monitoring device.

10. The person support apparatus according to claim 1, wherein the controller is further configured to transmit an alert to a caretaker device.

11. The person support apparatus according to claim 1, wherein the controller is further configured to:
receive an indication of an exit alarm setting;
determine, based on the positioning of the subject, that a bed exit event is occurring or likely to occur; and
transmit a bed exit alarm.

12. The person support apparatus according to claim 1, further comprising:
a fluid source; and
one or more pumps fluidly coupling the fluid source to the plurality of bladders, the one or more pumps communicatively coupled to the controller,
wherein the controller is further configured to transmit a signal to the one or more pumps to increase or decrease an internal pressure of each of the plurality of bladders.

13. The person support apparatus according to claim 12, wherein the controller is further configured to determine that at least one of the plurality of bladders is overinflated or underinflated, determine an inflation or deflation command, and transmit the signal to the one or more pumps in accordance with the inflation or deflation command.

14. The person support apparatus according to claim 1, wherein the positioning of the subject is selected from a laying position, an upright sitting position, a right side sitting position, and a left side sitting position.

15. The person support apparatus according to claim 1, wherein the person support surface is a mattress topper positioned over one or more bed components.

16. The person support apparatus according to claim 1, wherein the person support surface is an integrated support surface.

17. A system for determining subject positioning, the system comprising:
a plurality of pressure sensors, each one of the plurality of pressure sensors fluidly coupled to a corresponding one of a plurality of bladders on a person support surface;
a processor communicatively coupled to the plurality of pressure sensors; and
a non-transitory storage medium communicatively coupled to the processor, the non-transitory storage medium storing programming instructions thereon that, when executed, cause the processor to:
receive an input corresponding to an indication that a subject is supported on the person support surface,
receive a first signal from each one of the plurality of pressure sensors, the first signal indicative of an initial reference pressure of each of the plurality of bladders,
receive a subsequent signal from each of the plurality of pressure sensors after a predetermined period of time has elapsed, the subsequent signal indicative of a subsequent pressure of each of the plurality of bladders,
determine a rate of change in pressure based on a difference between the first signal and the subsequent signal for each of the plurality of bladders,
determine, based on the rate of change for each of the plurality of bladders, that a subject movement has occurred, and
determine, based on the subject movement, a positioning of the subject.

18. The system according to claim 17, wherein the plurality of bladders comprises a first head bladder, a second head bladder, a first seat bladder, and a second seat bladder.

19. The system according to claim 17, wherein the programming instructions, when executed, further cause the processor to:
receive data corresponding to a shape and a size of each of the plurality of bladders.

20. The system according to claim 19, wherein the programming instructions that, when executed, cause the processor to determine that the subject movement has occurred is further based on the shape and the size of each of the plurality of bladders.
